(19) United States
(12) Patent Application Publication
(10) Pub. No.: US 2009/0252801 A1
(43) Pub. Date: Oct. 8, 2009

(54) Process for the Preparation of Micronised Sterile Steroids

(75) Inventors: Giuseppe Fortunato, Trecate (IT); Claudio Gianluca Pozzoli, Monza (IT); Franco Ricci, Carenno (IT)

Correspondence Address: OHLANDT, GREELEY, RUGGIERO & PERLE, LLP, ONE LANDMARK SQUARE, 10TH FLOOR, STAMFORD, CT, 06901, US

(73) Assignee: FARMBIOS S.P.A., Gropello Cairoli (IT)

(21) Appl. No.: 12/085,602

(22) PCT Filed: Nov. 29, 2005
(86) PCT No.: PCT/EP2005/056307

§ 371 Date: May 28, 2008

(51) Int. Cl. A61K 9/14; A61K 31/56; A61P 15/18

(52) U.S. Cl. 424/489; 514/169

(57) ABSTRACT

The present invention relates to a process for the preparation of micronised sterile steroids, comprising sterilisation of the steroids in crystalline form by means of irradiation with gamma or beta rays, and subsequent sterile micronisation.

### FIELD OF THE INVENTION

The present invention refers to a process for preparing micronised sterile steroids, useful for the preparation of pharmaceutical compositions for inhalation administration in the treatment of asthma and allergic conditions and/or inflammatory conditions, as well as for the use as contraceptive or antineoplastic agents for parenteral administration.

### STATE OF THE ART

It is well known that a fundamental requirement for the pharmaceutical formulations, to be used for inhalation in the form of solutions, or suspensions supplied as pressurised aerosol (MDI) or by nebulisation (NEBUL), or by using suitable ultrasonic devices, or compressed air, is the sterility of the formulations; sterility is the main requirement for injectable suspensions as well.

Several methods for sterilising steroids are known, but each method presents limitations or inadequacies.

In general, according to these known methods, the active principle is subjected to a pre-sterilisation step, followed by preparation in asepsis, or alternatively, firstly the formulation is prepared then it is subjected to sterilisation by treatment in autoclave.

The pre-sterilisation methods require a subsequent step of mixing of the active principle with other components of the formulation, and then they require the final formulation in asepsis.

The standard treatments of autoclaving in the case of aqueous suspensions of thermolabile corticosteroids are not appropriate because they provoke the degradation of the active principle, or they originate re-aggregation phenomena of the active principle particles, that are then difficult to be separated and dispersed in the suspension, so that the therapeutic efficiency would be prejudiced, especially in case of aerosol therapy.

The sterilising filtration in case of suspensions is not practicable because it requires the use of filters having pores dimensions not higher than 0.2 μm that is much lower than the diameter of most of particles in the active principle, so that many of these particles remain in the filter.

Many methods have been proposed in the past for sterilising micronised steroids. The patent PT-A-69652 describes the cold sterilisation of micronised steroids with a mixture of ethylene oxide and carbon dioxide. In this patent are reported various examples of sterilisation of steroids, such as prednacindone, dexamethasone, prednisolone and esters thereof, fluoro derivatives and salts thereof, including dexamethasone acetate, dexamethasone phosphate, prednisolone pivalate and 9-alphafluoroprednisolone.

A fundamental limitation of this process consists in that ethylene oxide is toxic and the residue remaining inside the product after sterilisation of the steroid is high and not in accordance with the pharmaceutical guidelines (ICH), which require a very low residue of ethylene oxide in the final product. Therefore, in view of the recent regulatory requirements, this method would not be suitable for the manufacture of acceptable pharmaceutical formulations of steroids.

The U.S. Pat. No. 6,392,036 (Astra Zeneca) describes the sterilisation of steroids by heat treatment at a temperature ranging from 100 to 130° C. and for times ranging from 1 to 10 hours, according to the temperature and the steroids. The steroid is in the form of finely divided particles, having a diameter of from 10 to 5 μm, and it is substantially dry (the content of water is less than 1% w/w, preferably less than 0.5% w/w and more preferably less than 0.3% w/w). The bioburden before sterilisation is preferably of less than 1 CFU per gram. The process disclosed in U.S. Pat. No. 6,392,036 is scarcely useful for the industrial preparation of bulk-micronised steroids; in fact the scaled-up preparation requires a very complicated validation procedure of both the process and the dry oven steriliser, because it is necessary to prove that the sterilisation temperature is achieved everywhere inside the product and maintained for the time needed.

In U.S. Pat. No. 6,464,958 it is described a process for the preparation of sterile steroid powders (being in turn suitable for the preparation of pharmaceutical compositions for specified therapeutic use) wherein the solid crystalline active agent is micronised and thereafter sterilised by gamma irradiation.

A sterilisation process of steroids by irradiation with gamma or beta rays, and mainly with gamma rays, is also known in the art. A drawback of this process, in which irradiation is carried out on already micronised products, involves the critical alterations shown by the products subjected to this process: an increase in the total amount of degradation products with the formation of new degradation products has been observed (Illum and Moeller in Arch. Pharm. Chem. Sci.Ed. 2, 1974, pp. 167-174).

It is therefore evident the importance of developing a new process for preparing steroids in micronised sterile form, not having the drawbacks highlighted above for the processes known in the art.

### SUMMARY OF THE INVENTION

Now the Applicant has surprisingly found that the irradiation with beta or gamma rays, when carried out directly on non-micronised steroids in crystalline form, does not give rise to degradation processes and allows the physical-chemical characteristics of the products be preserved, even after sterile micronisation.

Subject of the present invention, is therefore a process for the preparation of a micronised sterile powder comprising a steroid or pharmaceutically acceptable ester or salt thereof, said process comprising the following steps:

i) sterilisation of a non-micronised powder comprising a steroid or pharmaceutically acceptable ester or salt thereof in crystalline form, by irradiation with beta or gamma rays;

ii) micronisation under sterile conditions of the sterile powder coming from step i). Further subject of the present invention is a micronised sterile powder comprising a steroid or pharmaceutically acceptable ester or salt thereof, obtainable by the above said process, in which said steroid contains less than 0.10% by weight of impurities not present in the starting product.

Further subjects of the invention are the use of the above said micronised sterile powder for the preparation of a pharmaceutical composition useful for the treatment of asthma and allergic conditions and/or inflammatory conditions of the nose or lungs and as contraceptive or antineoplastic agent; and a pharmaceutical composition comprising the above said micronised sterile powder as active principle.

Features and advantages of the present invention will be illustrated in detail in the following description.

### BRIEF DESCRIPTION OF THE DRAWINGS

FIG. 1: HPLC chromatograms (Absorbance in mV vs. time in min) of A=starting crystalline medroxyprogesterone acetate, B=sterile micronised medroxyprogesterone acetate obtained according to Example 1 of the invention, and C=sterile micronised medroxyprogesterone acetate obtained according to comparative Example 2.

FIG. 2: HPLC chromatograms (Absorbance in mV vs. time in min) of A=starting crystalline triamcinolone acetonide, B=sterile micronised triamcinolone acetonide obtained according to Example 3 of the invention, and C=sterile micronised triamcinolone acetonide obtained according to comparative Example 4.

### DETAILED DESCRIPTION OF THE INVENTION

The present invention makes it possible to meet the above mentioned requirements thanks to the use of gamma or beta rays for the sterilisation of steroids in non-micronised crystalline form, carrying out then the micronisation under sterile conditions.

According to the present process, the sterilisation procedure in step i) may be carried out on the powder product packed under vacuum in a suitable container, such as a sealed bag made of a suitable plastic material, preferably polyethylene; this container is in its turn sealed in another bag made of oxygen-proof materials, to avoid the presence of oxygen during irradiation.

The sterile product coming from step i) has to be then subjected to micronisation under sterile conditions, therefore the above said container is opened under aseptic conditions, the sterile product is introduced in a suitable apparatus, such as Jet Mill microniser, able to carry out micronisation under sterile conditions; after micronisation, the sterile micronised product may be packed again in a suitable container, preferably a polyethylene bag.

The sterilisation step i) according to the process of the invention was validated according to International Standard Organisation Procedure ISO 11137-2B to guarantee a Sterility Assurance Level SAL of at least 10⁻⁶, preferably of 10⁻⁷, and the so obtained product is “sterile” according to the criteria of European Pharmacopoeia and US Pharmacopoeia.

Generally, the starting non-micronised powder comprising the steroid in crystalline form is substantially dry; its content of water is typically lower than 1% by weight with respect to the total weight of the powder, preferably is lower than 0.5% and more preferably lower than 0.2%. Nevertheless, as starting non-micronised powder according to the invention, a known hydrated form of the steroid may also be used, such as for beclomethasone monohydrate and flunisolide hemihydrate.

According to the invention the starting non-micronised powder comprising the s steroid has typically a bioburden of less than 10 CFU (Colony Forming Units) per gram of product, and preferably of less than 1 CFU.

The sterilisation step i) of the present process is carried out by irradiating the starting non-micronised material to irradiation with beta or gamma rays, and preferably with gamma rays, at 1 to 25 KGy, and preferably at 4 to 10 KGy.

After irradiation, the resulting product is subjected to the micronisation step ii) of the present process under sterile conditions in a suitable microniser, able to maintain sterile conditions during working operations, such as the Jet mill apparatus, under a pressure ranging from 1 to 12 bar, preferably from 6 to 8 bar, using sterile air or nitrogen as fluid stream.

Moreover, the micronisation step ii) is carried out at a temperature ranging from 0 to 30° C., and preferably at a temperature ranging from 20 to 25° C. The so obtained micronised product has a particle size distribution between 1 and 30 μm, and preferably 99% of the particles have size equal or lower than 10 μm and 90% of the particles have size equal to or lower than 5 μm.

Examples of steroids and esters or salts thereof, which may be used in the present process, include medroxyprogesterone acetate, budesonide, triamcinolone acetonide, fluticasone propionate, triamcinolone diacetate, triamcinolone hexacetonide, momethasone furoato, beclomethasone, beclomethasone dipropionate, flunisolide, flurandrenolide and hydrocortisone acetate, in dry or hydrated form as said above.

It was proved by the Applicant that the present sterilisation procedure, when applied to non-micronised steroids in crystalline form, does not cause any degradation process and the amount of impurities present in the starting products do not increase or, when an increase is observed, it is lower than 0.10% by weight, preferably lower than or equal to 0.05%, and in any case the resulting non-micronised sterile product has a purity degree, determined by HPLC analysis, in accordance with the criteria of European and US Pharmacopoeia.

Moreover, it was also proved by means of DSC (Differential Scanning Calorimetry), TGA (Thermo Gravimetric Analysis) and XRD (X-Ray Diffractometry), that no modifications of the crystalline structure of the starting steroid occurs when it is subjected to the present sterilisation step.

Analogous results were obtained after the present micronisation step ii), by carrying out the same above mentioned analysis on the final micronised sterile product too.

The present process is therefore useful to obtain steroids in sterile micronised form, having high purity and containing less than 0.05% by weight of impurities not present in the starting product, so that, depending on the purity of the starting material, the present process is able to yield steroids having a purity degree of at least 99.5% by weight.

Furthermore, the present process allows to obtain a sterilised steroid having the same pharmacological activity, the same physical-chemical properties, the same crystalline features and substantially the same purity degree of the starting product; of particular relevance is the fact that the chemical degradation caused by exposure to gamma rays of the micronised product is a very limited phenomenon when the non-micronised product is subjected to this kind of sterilisation.

The present sterile micronised powder, comprising or consisting of the sterile micronised steroid prepared as described above, may be used for preparing sterile pharmaceutical compositions useful for the treatment of allergic conditions and/or inflammatory conditions of nose and lungs, such as rhinitis, asthma, chronic obstructive pulmonary diseases (chronic bronchitis and emphysema), and bronchopulmonary dysplasia.

The resulting pharmaceutical compositions, which may comprise one or more pharmaceutically acceptable excipients and/or diluents, are preferably in the form of aqueous suspensions suitable for aerosol inhalation and for parenteral administration. These sterile aqueous suspensions showed physical and chemical stability after long-term accelerated storage conditions.

The following examples are reported as a non limiting illustration of the invention.

### EXAMPLE 1

### Sterilisation of Crystalline Medroxyprogesterone Acetate Followed by Sterile Micronisation According to the Invention

500 g of crystalline medroxyprogesterone acetate are packed under vacuum in a polyethylene double bag, and sterilised by irradiation with gamma rays at 8 KGy. Then the product has been micronised under sterile conditions in a Jet Mill apparatus at 20° C. using nitrogen as fluid stream at a pressure of 7 bar, obtaining the sterile micronised medroxyprogesterone acetate as the final product in which 99% of the particles have size equal to or lower than 10 μm and 90% of the particles have size equal to or lower than 5 μm.

After both steps of irradiation with gamma rays and micronisation, the purity degree of the product and the amount of impurities contained were determined by HPLC. The so obtained results are shown in the following Table 1.

TABLE 1

| | Crystalline | Crystalline | Sterile |
|---|---|---|---|
| | starting | product sterilised | micronised |
| | material | with gamma-rays | final product |
| Purity | 99.64% | 99.63% | 99.64% |
| Known impurities | 0.34% | 0.35% | 0.33% |
| Unknown impurities | 0.02% | 0.02% | 0.03% |
| Total impurities | 0.36% | 0.37% | 0.36% |

The results reported above show that crystalline medroxyprogesterone acetate is stable after irradiation with gamma rays, as well as after the subsequent micronisation under sterile conditions, and the final sterile product in micronised form meets the desired requirements of purity.

The starting material, the crystalline product sterilised with gamma-rays and the sterile micronised final product, were all subjected to XRD, DSC and TGA analysis, to the determination of water content according to the Karl Fisher method, to the determination of: sulphuric ashes, melting point, rotatory power and residual solvents, thus finding that no significant modification occurred in the above said analysed features following to the application of the present sterilisation and micronisation procedures.

### EXAMPLE 2 (COMPARISON)

### Sterilisation of Micronised Medroxyprogesterone Acetate

500 g of medroxyprogesterone acetate, previously micronised in a Jet Mill apparatus at 20° C. using nitrogen as fluid stream at 7 bar so to obtain a micronised product in which 99% of the particles have size equal to or lower than 10 μm and 90% of the particles have size equal to or lower than 5 μm, have been packed under vacuum in a polyethylene double bag, and then subjected to irradiation with gamma rays at 8 KGy.

After the irradiation, the purity of the product and the amount of impurities were determined by HPLC. The so obtained results are shown in the following Table 2.

TABLE 2

| | Crystalline | Micronised | Sterile |
|---|---|---|---|
| | starting | starting | micronised |
| | material | material | final product |
| Purity | 99.64% | 99.63% | 99.05% |
| Known impurities | 0.34% | 0.34% | 0.62% |
| Unknown impurities | 0.02% | 0.03% | 0.33% |
| Total impurities | 0.36% | 0.37% | 0.95% |

The results summarised above in Table 2 show that the micronised medroxyprogesterone acetate, when subjected to the same amount of gamma rays exposure of 8 KGy, gives a significant amount of both the known and unknown impurities.

In the following Table 3 the relevant data obtained for the final products of above Examples 1 and 2, already reported in the third column of Table 1 and 2, are reported next to each other to highlight better the difference between the products in terms of purity; moreover, the same data obtained for the starting material are also reported again, to show the advantages of the present process.

TABLE 3

| | | Sterile | Sterile micronised |
|---|---|---|---|
| | Crystalline | micronised | final product obtained |
| | starting | final product | by gamma irradiation |
| | material | of the invention | of micronised product |
| Purity | 99.64% | 99.64% | 99.05% |
| Known impurities | 0.34% | 0.33% | 0.62% |
| Unknown impurities | 0.02% | 0.03% | 0.33% |
| Total impurities | 0.36% | 0.36% | 0.95% |

From the results reported above it is evident that non-micronised sterile medroxyprogesterone acetate, obtained by sterilisation with gamma rays of the crystalline starting product followed by micronisation under sterile conditions, does not show any significant increase in the amount of degradation products.

On the contrary, starting from the same initial product, but inverting the present steps of sterilisation and micronisation, i.e. carrying out irradiation on the product already micronised, a significant increase in the amount of degradation by-products was observed in the final micronised sterile steroid.

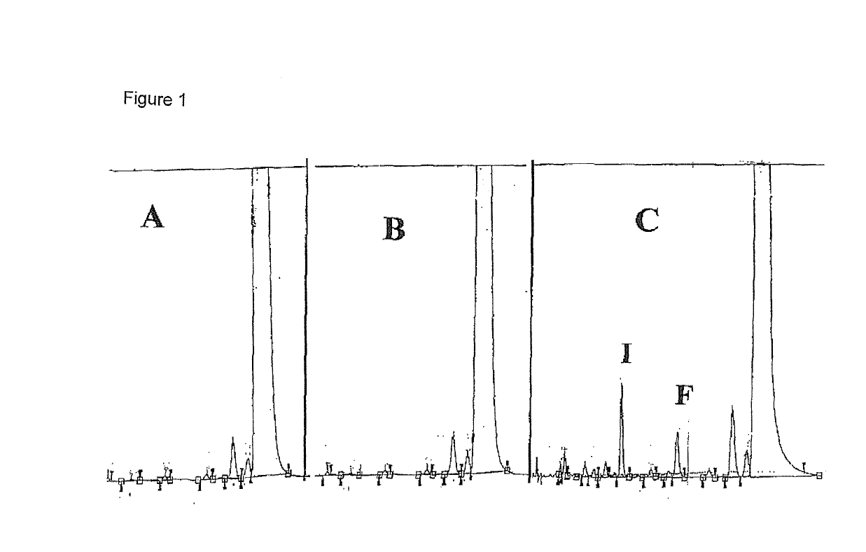

A self-explanatory showing of the above facts is represented by FIG. 1, wherein the HPLC chromatogram of the starting product (A) has been reported close to the chromatogram of the sterile micronised final product of the invention (B) as prepared in Example 1, and to the chromatogram of the comparative product obtained by sterilising the micronised product (C) of Example 2, wherein the two peaks indicated as “I” and “F” are clearly corresponding to two impurities not present either in the starting product (A) or in the final product of the invention (B).

### EXAMPLE 3

### Sterilisation of Crystalline Triamcinolone Acetonide Followed by Sterile Micronisation According to the Invention

500 g of crystalline triamcinolone acetonide are packed under vacuum in a polyethylene double bag, and sterilised by irradiation with gamma rays at 8 KGy. Then the product has been micronised under sterile conditions in a Jet Mill apparatus at 20° C. using nitrogen as fluid stream at 7 bar, obtaining the sterile micronised triamcinolone acetonide as the final product in which 99% of the particles have size equal to or lower than 10 μm and 70% of the particles have size equal to or lower than 5 μm.

After both steps of irradiation with gamma rays and micronisation, the purity degree of the product and the amount of impurities contained were determined by HPLC. The so obtained results are shown in the following Table 4.

TABLE 4

| | Crystalline | Crystalline | Sterile |
|---|---|---|---|
| | starting | product sterilised | micronised |
| | material | with gamma-rays | final product |
| Purity | 99.86% | 99.86% | 99.86% |
| Known impurities | 0.02% | 0.02% | 0.02% |
| Unknown impurities | 0.12% | 0.12% | 0.12% |
| Total impurities | 0.14% | 0.14% | 0.14% |

The results reported above show that crystalline triamcinolone acetonide is stable after irradiation with gamma rays, as well as after the subsequent micronisation under sterile conditions, and the final sterile product in micronised form meets the desired requirements of purity.

The starting material, the crystalline product sterilised with gamma-rays and the sterile micronised final product, were all subjected to XRD, DSC and TGA analysis, to the determination of water content according to the Karl Fisher method, to the determination of: sulphuric ashes, melting point, rotatory power and s residual solvents, thus finding that no significant modification occurred in the above said analysed features following to the application of the present sterilisation and micronisation procedures.

### EXAMPLE 4 (COMPARISON)

### Sterilisation of Micronised Triamcinolone Acetonide

500 g of triamcinolone acetonide, previously micronised in a Jet Mill apparatus at 20° C. using nitrogen as fluid stream at 7 bar, so to obtain a micronised product in which 99% of the particles have size equal to or lower than 10 μm and 70% of the particles have size equal to or lower than 5 μm, have been packed under vacuum in a polyethylene double bag, and then subjected to irradiation with gamma rays at 8 KGy.

After the irradiation, the purity of the product and the amount of impurities were determined by HPLC. The so obtained results are shown in the following Table 5.

TABLE 5

| | Crystalline | Micronised | Sterile |
|---|---|---|---|
| | starting | starting | micronised |
| | material | material | final product |
| Purity | 99.86% | 99.85% | 99.37% |
| Known impurities | 0.02% | 0.02% | 0.21% |
| Unknown impurities | 0.12% | 0.13% | 0.42% |
| Total impurities | 0.14% | 0.15% | 0.63% |

From the results reported above in Table 5 it is evident that the micronised triamcinolone acetonide, when subjected to the same amount of gamma rays exposure of the non-micronised product, show a significant increase in the amount of both known and unknown impurities.

In the following Table 6 the relevant data obtained for the final products of above Examples 3 and 4, already reported in the third column of Table 4 and 5, are reported next to each other to highlight better the difference between the products in terms of purity; moreover, the same data obtained for the starting material are also reported again, to show the advantages of the present process.

TABLE 6

| | | Sterile | Sterile micronised |
|---|---|---|---|
| | Crystalline | micronised | final product obtained |
| | starting | final product | by gamma irradiation |
| | material | of the invention | of micronised product |
| Purity | 99.64% | 99.86% | 99.37% |
| Known impurities | 0.34% | 0.02% | 0.21% |
| Unknown impurities | 0.02% | 0.12% | 0.42% |
| Total impurities | 0.36% | 0.14% | 0.63% |

From the results reported above it is evident that micronised sterile triamcinolone acetonide, obtained by sterilisation with gamma rays of the starting product in crystalline form followed by micronisation under sterile conditions, does not show any significant increase in the amount of degradation products.

On the contrary, starting from the same initial product, but inverting the present steps of sterilisation and micronisation, i.e. carrying out irradiation on the product already micronised, a significant increase in the amount of degradation by-products was observed in the final micronised sterile triamcinolone acetonide.

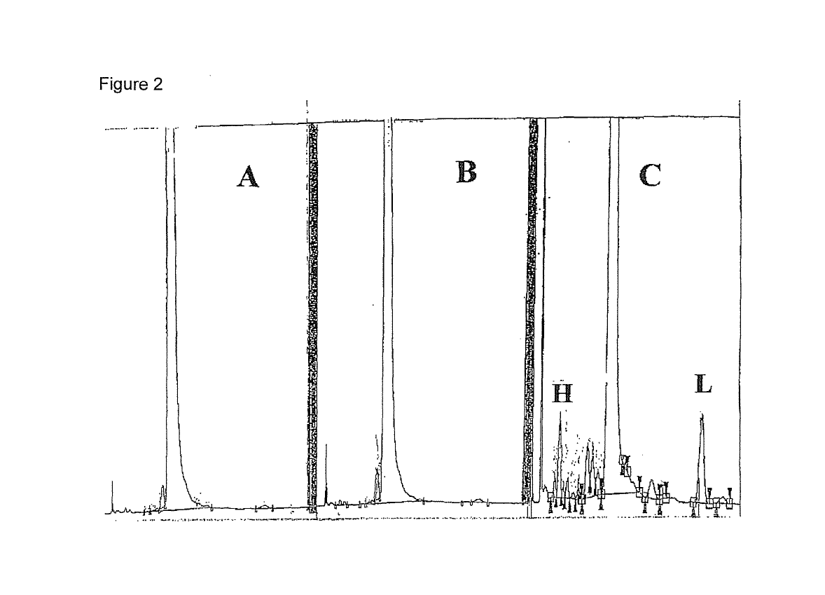

In FIG. 2 the HPLC chromatogram of the starting product (A) is reported close to the chromatogram of the sterile micronised final product of the invention (B) obtained in Example 3 and to the chromatogram of the comparative product obtained by sterilising the micronised product (C) in Example 4, wherein the two peaks indicated as “H” and “L” are clearly corresponding to two impurities not present either in the starting product (A) or in the final product of the invention (B). Moreover, the chromatogram (C) also shows many other smaller peaks not present in chromatograms (A) and (B).

1. A process for the preparation of a micronised sterile powder comprising a steroid or pharmaceutically acceptable ester or salt thereof, said process comprising the following steps:
i) sterilisation of a non-micronised powder comprising a steroid or pharmaceutically acceptable ester or salt thereof in crystalline form, by irradiation with beta or gamma rays;
ii) micronisation under sterile conditions of the sterile powder coming from step i).

2. The process according to claim 1, wherein said steroid or pharmaceutically acceptable ester or salt thereof is selected from the group consisting of medroxyprogesterone acetate, budesonide, triamcinolone acetonide, fluticasone propionate, triamcinolone diacetate, triamcinolone hexacetonide, momethasone furoato, beclomethasone, beclomethasone dipropionate, flunisolide, flurandrenolide and hydrocortisone acetate.

3. The process according to claim 1, wherein said sterilisation in step i) is carried out by irradiation with gamma rays.

4. The process according to claim 1, wherein said sterilisation in step i) is carried out by irradiation with gamma rays at 1 to 25 KGy.

5. The process according to claim 1, wherein said sterilisation in step i) is carried out by irradiation with gamma rays at 4 to 10 KGy.

6. The process according to claim 1, wherein said non-micronised powder is previously packed under vacuum in a suitable sealed container before irradiation in step i).

7. The process according to claim 6, wherein said suitable sealed container is made of polyethylene.

8. The process according to claim 1, wherein said micronisation in step ii) is carried out in a suitable microniser, able to maintain sterile conditions during working operations, at a pressure ranging from 1 to 12 bar, at a temperature ranging from 0 to 30° C., using sterile air or nitrogen as fluid stream.

9. The process according to claim 8, wherein said micronisation in step ii) is carried out at a pressure ranging from 6 to 8 bar, and at a temperature ranging from 20 to 25° C.

10. The process according to claim 1, wherein the final product obtained from micronisation in step ii) has the particles size distribution ranging between 1 and 30 μm.

11. The process according to claim 10, wherein in the final product obtained from micronisation in step ii) the 99% of the particles have size equal or lower than 10 μm and 90% of the particles have size equal to or lower than 5 μm.

12. A micronised sterile powder comprising a steroid or pharmaceutically acceptable ester or salt thereof, obtainable by the process as defined in claim 1, in which said steroid contains less than 0, 10% by weight of impurities not present in the starting product.

13. The micronised sterile powder according to claim 12, wherein said steroid contains not more than 0.05% by weight of impurities not present in the starting product.

14. A pharmaceutical composition for the treatment of allergic conditions and/or inflammatory conditions of the nose or lungs comprising a micronised sterile powder according to claim 12.

15. The pharmaceutical composition according to claim 14, wherein said allergic conditions and/or inflammatory conditions of the nose or lungs are selected from rhinitis, asthma, chronic obstructive pulmonary diseases (chronic bronchitis and emphysema), and bronchopulmonary dysplasia.

16. A pharmaceutical composition as contraceptive or antineoplastic agent lungs comprising a micronised sterile powder according to claim 12.

17. A pharmaceutical composition comprising as active principle the micronised sterile powder as defined in claims 12.

18. The pharmaceutical composition according to claim 17, further comprising pharmaceutically acceptable excipients and/or diluents.

19. The pharmaceutical composition according to claim 17, in the form of an aqueous suspension for aerosol inhalation or parenteral administration.

20. The pharmaceutical composition according to claim 19, wherein said aqueous suspension shows physical and chemical stability after long-term accelerated storage conditions.
